(19) United States
(12) Patent Application Publication
(10) Pub. No.: US 2007/0117102 A1
(43) Pub. Date: May 24, 2007

(54) Nucleotide analogs

(76) Inventor: Philip R. Buzby, Brockton, MA (US)

Correspondence Address: GOODWIN PROCTER LLP;PATENT ADMINISTRATOR, EXCHANGE PLACE, BOSTON, MA, 02109-2881, US

(21) Appl. No.: 11/286,516

(22) Filed: Nov. 22, 2005

(51) Int. Cl. C12Q 1/68; C07H 21/04

(52) U.S. Cl. 435/6; 536/25.32

(57) ABSTRACT

The invention provides nucleotide analogs for use in sequencing nucleic acid molecules.

### FIELD OF THE INVENTION

The invention relates to nucleotide analogs and methods for sequencing a nucleic acid using the nucleotide analogs.

### BACKGROUND

There have been many proposals to develop new sequencing technologies based on single-molecule measurements. For example, sequencing strategies have been proposed that are based upon observing the interaction of particular proteins with DNA or by using ultra high resolution scanned probe microscopy. See, e.g., Rigler, et al., J. Biotechnol., 86(3):161 (2001); Goodwin, P. M., et al., Nucleosides & Nucleotides, 16(5-6):543-550 (1997); Howorka, S., et al., Nature Biotechnol., 19(7):636-639 (2001); Meller, A., et al., Proc. Nat'l. Acad. Sci., 97(3):1079-1084 (2000); Driscoll, R. J., et al., Nature, 346(6281):294-296 (1990).

Recently, sequencing by synthesis methodology has been proposed that resulted in sequence determination, but not with consecutive base incorporation. See, Braslavsky, et al., Proc. Nat'l Acad. Sci., 100:3960-3964 (2003). An impediment to base-over-base sequencing has been the high linear data density of DNA (3.4 A/base), which is an obstacle to the development of a single-molecule DNA sequencing technology. Scanned probe microscopes have had difficulty demonstrating simultaneous resolution and chemical specificity needed to resolve individual detectably labeled bases. Furthermore, read-length is often limited because of the inability of nucleic acid polymerizing agents to incorporate detectably labeled nucleotides or nucleotide analogs due to the steric hinderance produced by the detectable label.

A need therefore exists for nucleotide analogs that produce less background noise, thereby increasing the resolving power of scanned probe microscopy and that also have reduced steric hindrance, thereby allowing the polymerizing agent to produce greater read-length from each template.

### SUMMARY OF THE INVENTION

The present invention provides nucleotide analogs and methods of using the nucleotide analogs in sequencing. Nucleotide analogs of the present invention produce less background noise and display less steric hindrance of the polymerizing agent, thereby increasing the resolving power of scanned probe microscopy and providing improved read-length during single molecule sequencing. Nucleotide analogs of the present invention also optionally incorporate features that allow the incorporation of one nucleotide analog to the primer per round of extension, even where more than one type of nucleotide analog is present in the reaction or where the template comprises a homopolymeric stretch of two or more bases.

In general, nucleotide analogs of the present invention comprise a removable detectable moiety which is attached to the nucleotide analog. The detectable moiety can be removed during addition of the nucleotide analog to the primer. In other embodiments, the detectable moiety can be removed after the nucleotide analog has been added to the primer. In addition, the detectable signal produced by the removable detectable moiety can be modulated, e.g., quenched. In one embodiment, the signal is modulated by a removable quenching moiety which is also attached to the nucleotide analog. The quenching moiety can be removed from the nucleotide analog during the addition of the nucleotide analog to the primer or can be removed after the nucleotide analog has been added to the primer.

Nucleotide analogs of the present invention also can include a non-bridging sulfur in place of an oxygen at the α phosphate of the nucleotide. Further optionally, the nucleotide analogs of the present invention can include a phosphate group in place of the hydroxyl group in the 3′ position of the nucleotide sugar.

The detectable moiety is removable by virtue of being removably attached to the base of the nucleotide or by being attached to the γ phosphate group of the nucleotide. The quenching moiety is removable by virtue of being removably attached to the base or by being attached to the γ phosphate group of the nucleotide, whichever group does not have the detectable moiety attached. Where the detectable moiety or the quenching moiety is removably attached to the base, the detectable moiety or quenching moiety is attached with a cleavable or a cleavable/extended linker.

In general, methods of using the nucleotide analogs of the present invention comprise exposing a target nucleic acid/primer duplex to one or more nucleotide analogs of the present invention and a polymerizing agent under conditions suitable to extend the primer in a template dependent manner. Generally, the primer is sufficiently complementary to at least a portion of the target nucleic acid to hybridize to the target nucleic acid and allow template-dependent nucleotide polymerization. The primer is extended by one or more bases.

In single molecule sequencing, the template nucleic acid molecule/primer duplex is immobilized on a solid support such that nucleotides added to the immobilized primer are individually optically resolvable. The primer can be attached to the solid support, thereby immobilizing the hybridized template nucleic acid molecule, or the template can be attached to the solid support thereby immobilizing the hybridized primer. The primer and template can be hybridized to each other prior to or after attachment of either the template or the primer to the solid support.

During template dependent addition of the nucleotide analog to the primer (also referred to herein as primer extension), the pyrophosphate group of the nucleotide analog is removed. Depending on the nucleotide analog used, the pyrophosphate will have either the detectable moiety or the quenching moiety attached. Therefore, removal of the pyrophosphate group of the nucleotide analog during nucleotide addition to the primer results in the removal of either the detectable moiety or the quenching moiety of the nucleotide analog, respectively. Unincorporated nucleotide analogs are optionally removed from the template nucleic acid molecule/primer duplexes, e.g., by washing.

Each nucleotide analog added to the primer (if any) is identified by detecting the detectable moiety that is removably attached to the incorporated base or by detecting the detectable moiety that is attached to the released pyrophosphate group. The extended primer is then treated such that each remaining detectable moiety or quenching moiety, respectively (if any) is removed from the base. In certain embodiments, no nucleotide analog will have been added to the primer for example, where the nucleotide analog is not complementary to the target nucleotide.

Where the quenching moiety is attached to the base, the incorporated nucleotide analog can be detected before, during, or after the removal of the quenching moiety from the base because the label is present on the released pyrophosphate.

Where an optional phosphate group is present in place of the hydroxyl in the 3′ position of the nucleotide sugar, the optional phosphate moiety can be removed enzymatically. The incorporated nucleotide analog can be detected before, during, or after removing the optional phosphate group.

The primer extension process can be repeated to identify additional nucleotides in the template. The sequence of the template can determined by compiling the detected nucleotides, thereby determining the complimentary sequence of the target nucleic acid molecule.

The use of a removable detectable moiety reduces the background, allowing more sensitive detection of incorporated nucleotides and longer read-length. Removable detectable and quenching moieties also reduce the steric hindrance between the primer and the polymerizing agent. By removing the bulky detectable and/or quenching moiety, the polymerizing agent can add additional nucleotides or nucleotide analogs to the primer in subsequent rounds of primer extension, thereby producing longer read-length from each template nucleic acid. The combination of a detectable moiety and removable detectable and quenching moieties with promiscuous polymerases can further increase read-length.

In addition, optional phosphate group on the hydroxyl group at the 3′ position of the nucleotide sugar causes the nucleotide analog to act as a temporary terminator, preventing further addition of nucleotides to the primer. The use of a temporary terminator allows only one nucleotide to be added per round of primer extension even where the template comprises a homopolymeric stretch of two or more bases in length or when nucleotide analogs representing more than one class of base (e.g., A, G, C, T, or U) are added. Homopolymeric regions of sequence have been difficult to sequence using single molecule sequencing because of the difficulty in interpreting signal from the incorporation of multiple labeled nucleotides in a single round of extension. By using a temporary terminator, only one nucleotide analog will be added, preserving the usability of templates with homopolymeric regions. In addition, protecting the sugar allows the addition of nucleotide analogs corresponding to two or more of the four bases to the sequencing reaction at once; each labeled, for example, with a different detectable moiety. Where each nucleotide analog is a temporary terminator, a single nucleotide, complementary to the template portion of the duplex, will be added to each primer. Further additions to the primer are prevented until the phosphate group is removed. This should theoretically increase the rate of sequencing up to four-fold as well as increase the accuracy at which nucleotide repeats are read.

The nucleotide analogs of the present invention also optionally include a sulfur in place of a non-bridging oxygen of the a phosphate group. The presence of a sulfur in place of a non-bridging oxygen of the a phosphate group is expected to cause the nucleotide analog and polynucleotide comprising one or more of such nucleotide analogs to be resistant to nuclease activity, particularly nuclease activity that may be associated with enzymes used to remove the optional phosphate group in place of the hydroxyl group at the 3′ position of the nucleotide sugar.

While the invention is exemplified herein with fluorescent labels, the invention is not so limited and can be practiced using nucleotides labeled with any form of detectable label, including chemo luminescent labels, luminescent labels, phosphorescent labels, fluorescence polarization labels, and charge labels.

A detailed description of the certain embodiments of the invention is provided below. Other embodiments of the invention are apparent upon review of the detailed description that follows.

### BRIEF DESCRIPTION OF THE DRAWINGS

FIG. 1 is a generic chemical structure of the nucleotide analog of the present invention having an extended linker attached to the base.

FIG. 2 is a generic chemical structure of the nucleotide analog of the present invention having a linker attached to the base.

### DETAILED DESCRIPTION OF THE INVENTION

The invention is drawn generally to nucleotide analogs that, when used in sequencing reactions, have less background and allow greater read-length of the template nucleic acid molecule. Nucleotide analogs of the present invention include nucleotides comprising any one of the five standard bases, adenine, guanine, cytosine, thymine, or uracil, linked to a ribose or deoxyribose sugar which is linked to a triphosphate group, modified as described herein. The nucleotide analogs of the present invention also can comprise analogs of the five standard bases, as provided below.

Nucleotide Analogs

The nucleotide analogs of the present invention have the structure: 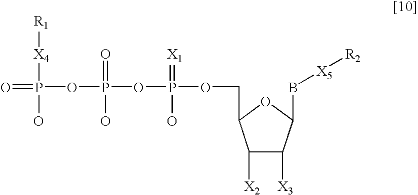
where X₁ can be O or S, X₂ can be OH or PO₄, and X₃ can be H or OH. Furthermore, nucleotide analogs of the present invention comprise a detectable moiety, D, and a quenching moiety, Q. In one embodiment, R₁ comprises the quenching moiety Q attached to the γ phosphate via X₄, X₄ being O, N or S and R₂ comprises the detectable moiety D attached to the base B via a cleavable linker X₅. In an another embodiment, R₁ comprises the detectable moiety D attached to the γ phosphate via X₄, and R₂ comprises the quenching moiety Q attached to the base via a cleavable linker X₅. The base B is a purine, deazapurine, pyrimidine, or derivative thereof.

The base B can be, for example, adenine, cytosine, guanine, thymine, uracil, or hypoxanthine. The base B can also be, for example, naturally-occurring and synthetic derivatives of the preceding group, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. Bases useful according to the invention permit a nucleotide that includes that base to be incorporated into a polynucleotide chain by a polymerizing agent and will form base pairs with a base on an antiparallel nucleic acid strand. The term base pair encompasses not only the standard AT, AU or GC base pairs, but also base pairs formed between nucleotides and/or nucleotide analogs comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the nucleotide analog inosine and adenine, cytosine or uracil, where the two hydrogen bonds are formed.

The detectable moiety can be, for example, a fluorophore. Preferred fluorophores include fluorescein, derivatives of fluorescein, BODIPY, derivatives of BODIPY, 5-(2′-aminoethyl)-aminonaphthalene-1-sulfonic acid (EDANS), rhodamine, derivatives of rhodamine, Cy2, Cy3, Cy3.5, CyS, Cy5.5, Texas Red and derivatives of Texas Red. Fluorophores can also be used as quenching moieties. Preferably, the quenching moiety is a non-fluorescent molecule, for example, a non-fluorescent aromatic or heteroaromatic moiety. In one embodiment, the quenching moiety is 4-((4-(dimethylamino)phenyl)azo) benzoic acid (DABCYL).

Modulation of the signal from the detectable moiety can comprise partial reduction in signal or complete reduction in signal from the detectable moiety. The reduction in signal from the moiety when present attached to the nucleotide analog, where the quenching moiety is also attached as described above, is at least about 80%. In other embodiments, the reduction in signal is at least about 90%, at least about 95%, or at least about 99%. The modulation of signal from the detectable moiety can occur through collision of detectable and quenching moieties that are closely associated by virtue of being attached to the same nucleotide analog. Modulation of signal from the detectable moiety also can occur through a nonradiative process such as fluorescence resonance energy transfer (FRET). For FRET to occur, transfer of energy between the detectable and quenching moieties requires that the moieties be close in space and that the emission spectrum of a detectable moiety has substantial overlap with the absorption spectrum of the quenching moiety (See: Yaron, et al. Anal. Biochem., 95:228-235 (1979) and particularly page 232, col. 1 through page 234, col. 1). Alternatively, collision mediated (radiationless) energy transfer may occur between very closely associated detectable and quenching moieties whether or not the emission spectrum of a detectable moiety has a substantial overlap with the absorption spectrum of the quenching moiety (See: Yaron, et al., Anal. Biochem., 95:228-235 (1979) and particularly page 229, col. 1 through page 232, col. 1).

As described above, the detectable moiety or the quenching moiety is linked to the base via a cleavable linker. The cleavable linker can be either a chemically cleavable linker or a photochemically cleavable linker. Chemically cleavable linkers can be cleaved under acidic, basic, oxidative, or reductive conditions. Examples of chemically cleavable linkers are provided below. In one embodiment, the chemically cleavable linker is a disulfide bond. Suitable photochemically cleavable linkages are provided below.

As described above, the nucleotide analogs of the present invention can also include a moiety at the 3′ position of the nucleotide sugar that prevents further extension of the primer after the nucleotide analog has been added to the primer. In one embodiment, the 3′ position of the nucleotide sugar has a phosphate group in place of the hydroxyl group. In order to prevent or reduce degradation of the primer containing the nucleotide analog or degradation of the nucleotide analogs, the nucleotide analog can further comprise a non-bridging sulfur on the a phosphate group of the nucleotide. The presence of the thiol group at the a phosphate position is expected to significantly improve the stability of the nucleotide analog as well as primers comprising one ore more nucleotide analogs, especially when exposed to enzymes capable of removing the optional phosphate group.

Nucleic Acid Sequencing

The present invention also includes methods for nucleic acid sequence determination using the nucleotide analogs described herein. The nucleotide analogs of the present invention are particularly suitable for use in single molecule sequencing techniques. Such techniques are described for example in U.S. patent application Ser. No. 10/831,214 filed Apr. 2004; 10/852,028 filed May 24, 2004; 10/866,388 filed Jun. 10, 2005; 10/099,459 filed Mar. 12, 2002; and U.S. Published Application 2003/013880 published Jul. 24, 2003, the teachings of which are incorporated herein by reference in their entireties. In general, the methods for nucleic acid sequence determination comprise exposing a target nucleic acid (also referred to herein as template nucleic acid or template) to a primer that is complimentary to at least a portion of the target nucleic acid, under conditions suitable for hybridizing the primer to the target nucleic acid, forming a template/primer duplex.

Target nucleic acids include deoxyribonucleic acid (DNA) and/or ribonucleic acid (RNA). Target nucleic acid molecules can be obtained from any cellular material, obtained from an animal, plant, bacterium, virus, fungus, or any other cellular organism. Target nucleic acids may be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid molecules may also be isolated from cultured cells, such as a primary cell culture or a cell line. The cells from which target nucleic acids are obtained can be infected with a virus or other intracellular pathogen.

A sample can also be total RNA extracted from a biological specimen, a cDNA library, or genomic DNA. Nucleic acid typically is fragmented to produce suitable fragments for analysis. In one embodiment, nucleic acid from a biological sample is fragmented by sonication. Test samples can be obtained as described in U.S. Patent Application 2002/0,190,663 A1, published Oct. 9, 2003, the teachings of which are incorporated herein in their entirety. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, target nucleic acid molecules can be from about 5 bases to about 20 kb. Nucleic acid molecules may be single-stranded, double-stranded, or double-stranded with single-stranded regions (for example, stem- and loop-structures).

One or more nucleotide analogs as described herein and a polymerizing agent are added to the template/primer duplex, under conditions suitable for extending the primer in a template-dependant manner. The primer can be extended by one or more nucleotide analogs. The addition of the nucleotide analog to the primer results in the removal of the terminal two phosphate groups with R₁ attached. The incorporated nucleotide analog is identified.

Where R₁ comprises the quenching moiety Q, the incorporated nucleotide analog is identified by detecting the detectable moiety D, attached to the base B via X₅. Unincorporated nucleotide analog molecules are removed prior to or after detecting. Unincorporated nucleotide analog molecules can be removed by washing. Where R₁ comprises the detectable moiety D, the incorporated nucleotide analog is identified by detecting the detectable moiety attached to the released pyrophosphate. In one embodiment, the reaction mixture containing the released pyrophosphate group is removed from the attached template/primer duplexes and the label of the detectable moiety is detected.

The template/primer duplex is then treated such that any detectable moiety or quenching moiety present on the incorporated nucleotide analog is removed as described below. As discussed herein, the detectable moiety can be modulatable. The steps of exposing template/primer duplex to one or nucleotide analogs and polymerizing agent, detecting incorporated nucleotides, and then treating to remove the remaining detectable or quenching moiety can be repeated, thereby identifying additional bases in the template nucleic acid, the identified bases can be compiled, thereby determining the sequence of the target nucleic acid. In some embodiments, the remaining detectable or quenching moiety is not removed, for example, in the last round of primer extension.

The R₂ group can be removed chemically or photochemically. In one embodiment, the cleavable linker X₅ is a photochemically cleavable linker, and the R₂ group is removed by exposing the extended primer to light of a suitable wavelength and of a suitable duration of time to cleave the photochemical linker, thereby causing the removable of the R₂ group from the incorporated nucleotide analog.

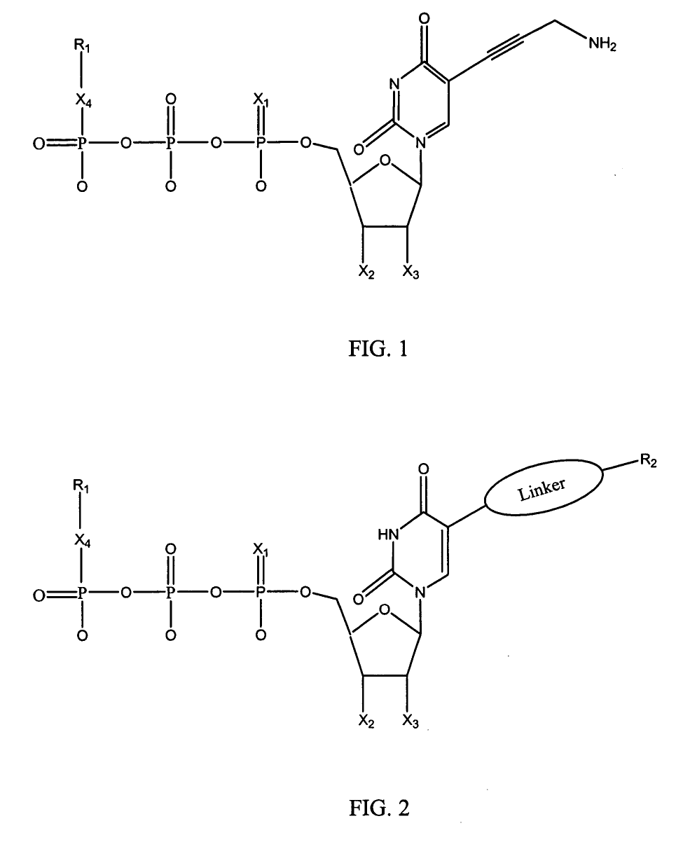

In one embodiment, an extended cleavable linker and fluorescent dye is used (Scheme 1). In this scenario, once the nucleotide analog is added to the primer, the fluorophore and linker can be removed by a photo-induced or chemically triggered cleavage. Once the bulky fluorophore is removed, it is anticipated that a less sterically encumbered system will result and, therefore, higher polymerase efficiency. Although uridine is shown as an example, all bases (A, U, C, G) and analogs thereof are included in this invention as described above. Also, although Scheme 1 shows a derivative of FIG. 1, where the base is uracil, any suitable base or derivative thereof can be used as described herein.

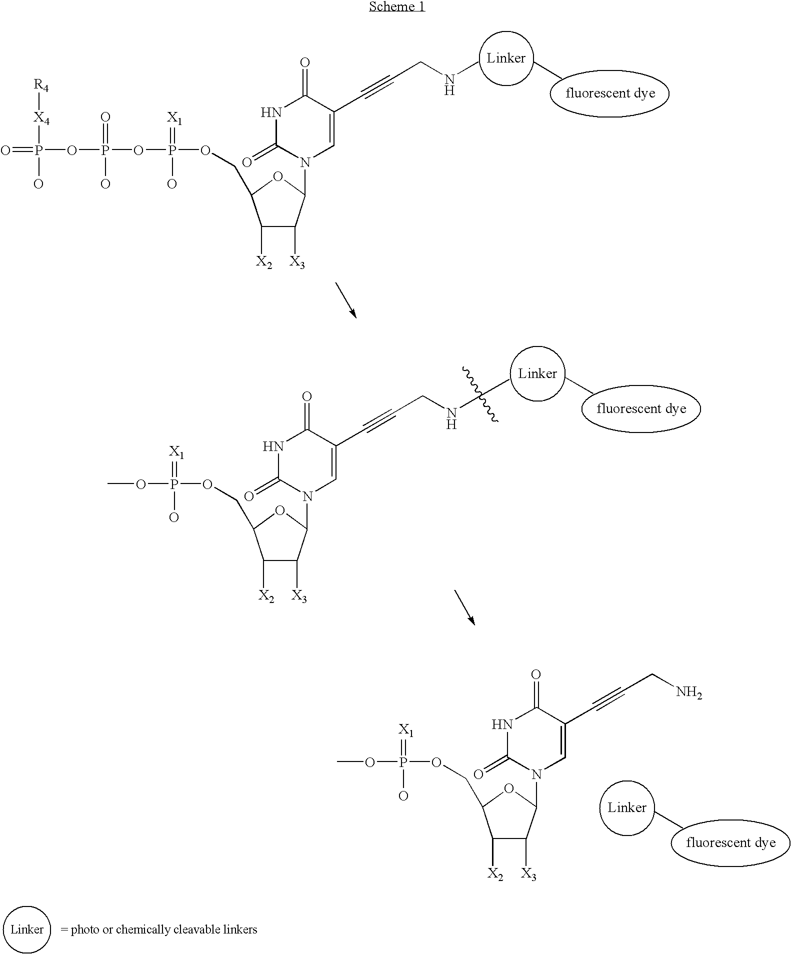

In another embodiment, the cleavable linker is attached directly to the base B as shown in Scheme 2. Scheme 2 shows a derivative of FIG. 2, where the base is uracil, however, as described herein, any suitable base or derivative thereof can be used.

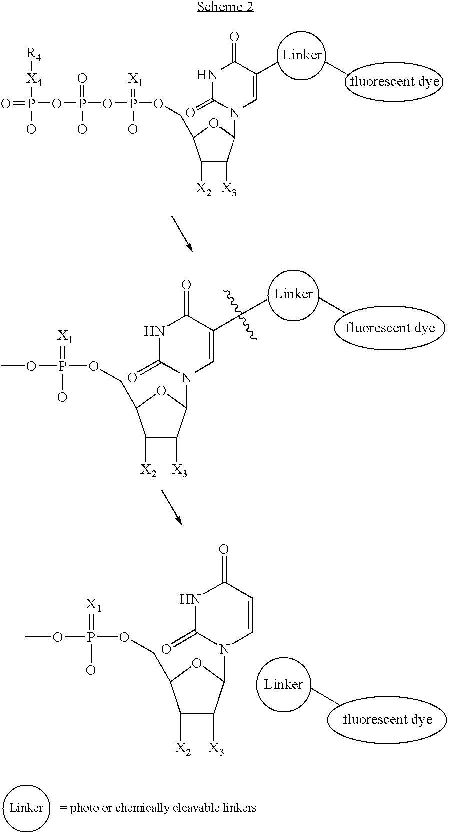

In one embodiment, the linker is a 2-nitrobenzyl linker. The 2-nitrobenzyl linker can be cleaved by photolysis at 340 nm. Polymerizing agents such as DNA polymerase can incorporate a nucleotide analog containing a 2-nitrobenzyl linker bridging a fluorophore and the base. Examples of additional molecules suitable for use as photochemical linkers are provided below (16-19): 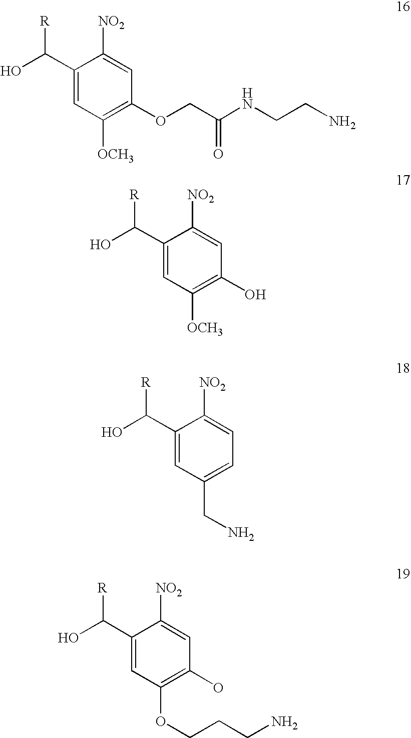
R═H, any chemical chain.

In other embodiments, the R₂ group comprises a detectable moiety or a quenching moiety attached to the base via a chemically cleavable bond. For example, amino acid and hydroxy acid derivatives can be used because they allow for the rapid synthesis of multiple nucleotide analogs through simple amide and ester bond forming reactions. However, this invention is not limited to amino acid and hydroxy acid derivatives. Any chemically removable linker is included in this invention.

Depending on the linker, chemically cleavable linkers can be cleaved under acidic, basic, oxidative, or reductive conditions. Where the cleavable linker comprises a chemically cleavable linker that is cleaved under reductive conditions, the primers having the nucleotide analog incorporated therein can be treated with, e.g., TCEP (tris(2-carboxyethyl) phosphine hydrochloride), β-mercaptoethanol, or DTT (dithiothreitol). In one embodiment, the cleavable linker is reduced, thereby releasing the detectable moiety or quenching moiety from the base of the nucleotide analog. Optionally, the cleaved or reduced linker is treated with an agent that renders the remaining portion of the linker non-reactive. For example, where the linker is a disulfide bond cleaved with a reducing agent, a sulfhydryl capping agent can be used to render the sulfer remaining on the nucleotide analog non-reactive. The sulfhydryl capping agent can be an alkylating agent such as iodoacetamide.

In another embodiment, amino acid 25 or commercially available alcohol 24 can be linked to a fluorophore and then cleaved by either base or enzyme-promoted hydrolysis of the ester bond. Another base-labile linker is 26, which has similar reactivity to the FMOC (fluorenylmethoxycarbonyl) protecting group. Amino acid linkers 27 and 28 will allow for removal of the fluorescent dye under acidic conditions as the acetal moieties can be gently hydrolyzed. Alternatively, α-substituted pentenoic acid derivative 29 will promote the liberation of the fluorophore under oxidative iodolactonization conditions, while the disulfide functionality of 30 and 31 will provide a substrate suitable for reductive cleavage. A linker diene 32 allows for release of the fluorophore under aqueous ring closing metathesis conditions. A linker 33 is removed after activation, for example with dithiothreitol.
Removed Under Basic Conditions 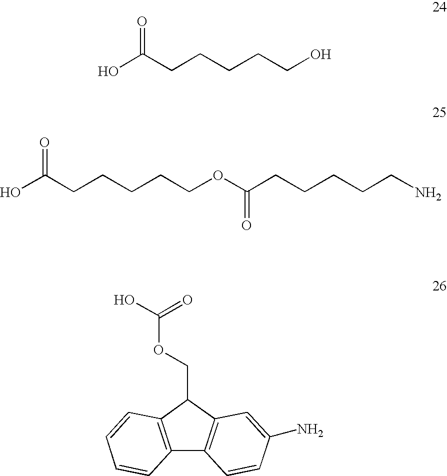
Removed Under Acidic Conditions 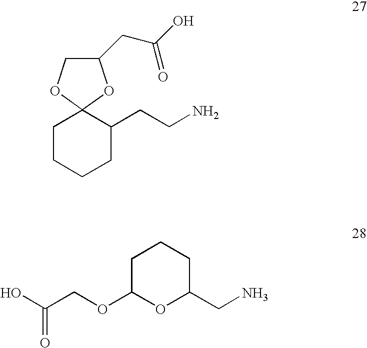
Removed Under Oxidative Conditions 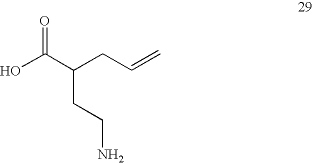
Removed Under Reductive Conditions 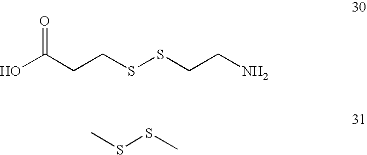
Removed Under Aqueous Ring Closing Metahesis Conditions 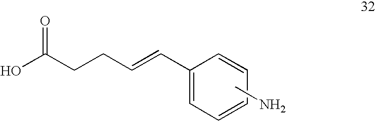
Removed Upon Activation

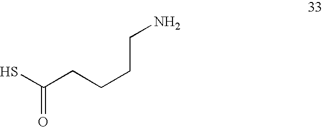

After addition of the nucleotide analog to the primer, the optional phosphate can be removed enzymatically. In one embodiment, the optional phosphate is removed using alkaline phosphatase or T₄ polynucleotide kinase. Suitable enzymes for removing the optional phosphate include, any phosphatase, for example, alkaline phosphatase such as shrimp alkaline phosphatase, bacterial alkaline phosphatase, or calf intestinal alkaline phosphatase.

Detection

Any detection method may be used to identify the incorporated nucleotide analog that is suitable for the type of label employed. Thus, exemplary detection methods include radioactive detection, optical absorbance detection, e.g., UV-visible absorbance detection, optical emission detection, e.g., fluorescence or chemiluminescence. Single-molecule fluorescence can be made using a conventional microscope equipped with total internal reflection (TIR) illumination. The detectable moiety associated with the extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Aca. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached target nucleic acids.

The present invention provides for detection of molecules from a single nucleotide to a single target nucleic acid molecule. A number of methods are available for this purpose. Methods for visualizing single molecules within nucleic acids labeled with an intercalating dye include, for example, fluorescence microscopy. For example, the fluorescent spectrum and lifetime of a single molecule excited-state can be measured. Standard detectors such as a photomultiplier tube or avalanche photodiode can be used. Full field imaging with a two-stage image intensified COD camera also can be used. Additionally, low noise cooled CCD can also be used to detect single fluorescent molecules.

The detection system for the signal may depend upon the labeling moiety used, which can be defined by the chemistry available. For optical signals, a combination of an optical fiber or charged couple device (CCD) can be used in the detection step. In those circumstances where the substrate is itself transparent to the radiation used, it is possible to have an incident light beam pass through the substrate with the detector located opposite the substrate from the target nucleic acid. For electromagnetic labeling moieties, various forms of spectroscopy systems can be used. Various physical orientations for the detection system are available and discussion of important design parameters is provided in the art.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophore identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for two-dimensional imaging. TIRF microscopy uses totally internally reflected excitation light and is well known in the art. See, e g., the World Wide Web at nikon-instrumentsjp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached target nucleic acid target molecule/primer complex in the presence of a polymerase. Total internal reflectance fluorescence microscopy is then used to visualize the attached target nucleic acid target molecule/primer complex and/or the incorporated nucleotides with single molecule resolution.

Fluorescence resonance energy transfer (FRET) can be used as a detection scheme. FRET in the context of sequencing is described generally in Braslavasky, et al., Proc. Nat'l Acad. Sci., 100:3960-3964 (2003), incorporated by reference herein. Essentially, in one embodiment, a donor fluorophore is attached to the primer, polymerase, or template. Nucleotides added for incorporation into the primer comprise an acceptor fluorophore that is activated by the donor when the two are in proximity.

Measured signals can be analyzed manually or by appropriate computer methods to tabulate results. The substrates and reaction conditions can include appropriate controls for verifying the integrity of hybridization and extension conditions, and for providing standard curves for quantification, if desired. For example, a control nucleic acid can be added to the sample. The absence of the expected extension product is an indication that there is a defect with the sample or assay components requiring correction.

In one embodiment, the detectable moiety is attached to the pyrophosphate group, and the pyrophosphate group is removed from the nucleotide analog during primer extension. The pyrophosphate containing the detectable moiety can be removed from the template/primer duplexes into a detection all where the presence and/or amount of the detectable label is determined, for example, by excitation at a suitable wavelength and detecting the fluorescence.

The present invention also includes methods of making the nucleotide analog. Syntheses of 2-nitrobenzyl linkers are known in the art. For example, linker 16 can be synthesized from the acid 20 through a DCC (N,N′-dicyclohexylcarbodiimide)-mediated coupling with ethylene diamine, followed by reduction of the ketone functionality (Scheme 3). Amino alcohol 16 can then be converted to photocleavable labeled dNTP 21, via two successive peptide bond forming reactions. Although synthesis of dUTP is shown, the other bases can be used, as well as ribonucleotides.

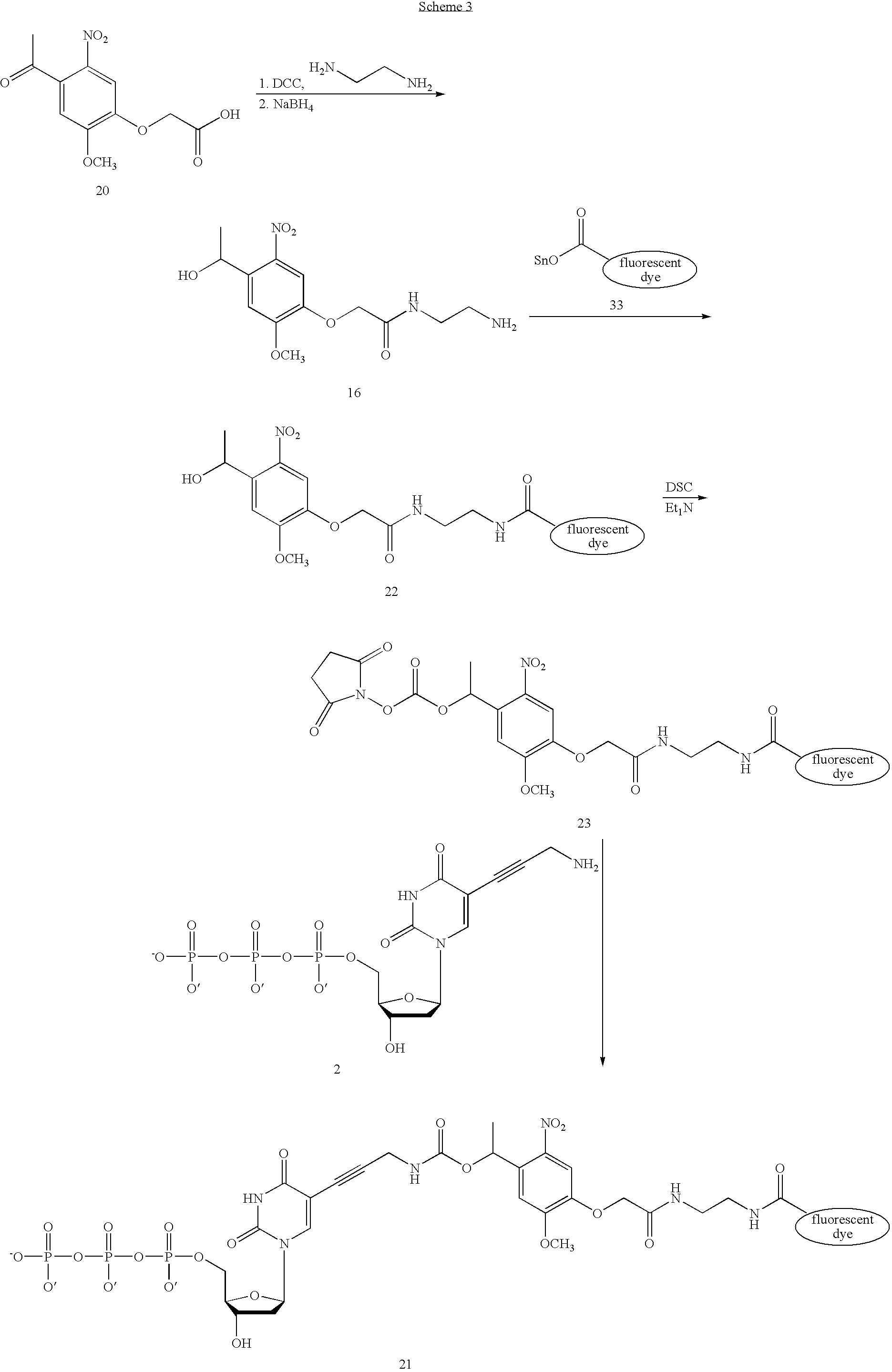

### Example

The 7249 nucleotide genome of the bacteriophage M13mp18 is sequenced using nucleotide analogs of the invention.

Purified, single-stranded viral M13mp18 genomic DNA was obtained from New England Biolabs. Approximately 25 ug of M13 DNA was digested to an average fragment size of 40 bp with 0.1 U Dnase I (New England Biolabs) for 10 minutes at 37° C. Digested DNA fragment sizes were estimated by running an aliquot of the digestion mixture on a precast denaturing (TBE-Urea) 10% polyacrylamide gel (Novagen) and staining with SYBR Gold (Invitrogen/Molecular Probes). The DNase I-digested genomic DNA was filtered through a YM10 ultrafiltration spin column (Millipore) to remove small digestion products less than about 30 nt. Approximately 20 pmol of the filtered DNase I digest was then polyadenylated with terminal transferase according to known methods (Roychoudhury, R and Wu, R. 1980, Terminal transferase-catalyzed addition of nucleotides to the 3′ termini of DNA. Methods Enzymol. 65(1):43-62). The average dA tail length was 50+/−5 nucleotides. Terminal transferase was then used to label the fragments with Cy3-dUTP. Fragments were then terminated with dideoxyTTP (also added using terminal transferase). The resulting fragments were again filtered with a YM10ultrafiltration spin column to remove free nucleotides and stored in ddH₂O at −20° C.

Epoxide-coated glass slides were prepared for oligo attachment. Epoxide-functionalized 40 mm diameter #1.5 glass cover slips (slides) were obtained from Erie Scientific (Salem, N.H.). The slides were preconditioned by soaking in 3×SSC for 15 minutes at 37° C. Next, a 500 pM aliquot of 5′ aminated polydT(50) (polythymidine of 50 bp in length with a 5′ terminal amine) was incubated with each slide for 30 minutes at room temperature in a volume of 80 ml. The resulting slides had poly(dT50) primer attached by direct amine linkage to the epoxide. The slides were then treated with phosphate (1 M) for 4 hours at room temperature in order to passivate the surface. Slides were then stored in polymerase rinse buffer (20 mM Tris, 100 mM NaCl, 0.001% Triton® X-100 (polyoxyethylene octyl phenyl ether), pH 8.0) until they were used for sequencing.

For sequencing, the slides were placed in a modified FCS2 flow cell (Bioptechs, Butler, Pa.) using a 50 um thick gasket. The flow cell was placed on a movable stage that is part of a high-efficiency fluorescence imaging system built around a Nikon TE-2000 inverted microscope equipped with a total internal reflection (TIR) objective. The slide was then rinsed with HEPES buffer with 100 mM NaCl and equilibrated to a temperature of 50° C. An aliquot of the M13 template fragments described above was diluted in 3×SSC to a final concentration of 1.2 nM. A 100 ul aliquot was placed in the flow cell and incubated on the slide for 15 minutes. After incubation, the flow cell was rinsed with 1×SSC/HEPES/0.1% SDS followed by HEPES/NaCl. A passive vacuum apparatus was used to pull fluid across the flow cell. The resulting slide contained M13 template/oligo(dT) primer duplex. The temperature of the flow cell was then reduced to 37° C. for sequencing and the objective was brought into contact with the flow cell.

For sequencing, cytosine triphosphate analog, guanidine triphosphate analog, adenine triphosphate analog, and uracil triphosphate analog, each having a fluorescent label, such as a Cy5, attached to the base via a cleavable linker, a quenching moiety, such as DABCYL, attached to the γ phosphate via O, S, or N, an optional S in place of a non-bridging O in the α phosphate, and an optional phosphate group in place of the OH group in the 2 position of the sugar, are stored separately in buffer containing 20 mM Tris-HCl, pH 8.8, 10 mM MgSO₄, 10 mM (NH₄)₂SO₄, 10 mM HCl, and 0.1% Triton® X-100 (polyoxyethylene octyl phenyl ether), and 100U Klenow exo⁻ polymerase (NEN). Sequencing proceeds as follows.

First, initial imaging is used to determine the positions of duplex on the epoxide surface. The Cy3 label attached to the M13 templates is imaged by excitation using a laser tuned to 532 nm radiation (Verdi V-2 Laser, Coherent, Inc., Santa Clara, Calif.) in order to establish duplex position. For each slide only single fluorescent molecules imaged in this step are counted. Imaging of incorporated nucleotides as described below is accomplished by excitation of a cyanine-5 dye using a 635 nm radiation laser (Coherent). 5 uM of a Cy5-labeled CTP analog as described above is placed into the flow cell and exposed to the slide for 2 minutes. For any Cy5-labeled CTP analogs that are incorporated into the primer, the enzymatic incorporation of the CTP analog results in the removal of the pyrophosphate moiety with the quenching moiety attached. After incubation, the slide is rinsed in 1×SSC/15 mM HEPES/0.1% SDS/pH 7.0 (“SSC/HEPES/SDS”) (15 times in 60 ul volumes each, followed by 150 mM HEPES/150 mM NaCl/pH 7.0 (“HEPES/NaCl”) (10 times at 60 ul volumes)). An oxygen scavenger containing 30% acetonitrile and scavenger buffer (134 ul HEPES/NaCl, 24 ul 100 mM Trolox in MES, pH 6.1, 10 ul DABCO in MES, pH 6.1, 8 ul 2M glucose, 20 ul Nal (50 mM stock in water), and 4 ul glucose oxidase) is next added. The slide is then imaged (500 frames) for 0.2 seconds using an Inova301K laser (Coherent) at 647 nm, followed by green imaging with a Verdi V-2 laser (Coherent) at 532 nm for 2 seconds to confirm duplex position. The positions having detectable fluorescence are recorded. After imaging, the flow cell is rinsed 5 times each with SSC/HEPES/SDS (60 ul) and HEPES/NaCl (60 ul).

Next, the fluorescent label, (e.g., the cyanine-5) is cleaved off of the incorporated CTP analogs. Where the cleavable linker is a disulfide bond, the Cy5 label is removed by introduction into the flow cell of 50 mM TCEP for 5 minutes, after which the flow cell was rinsed 5 times each with SSC/HEPES/SDS (60 ul) and HEPES/NaCl (60 ul), and the remaining nucleotide is capped with 50 mM iodoacetamide for 5 minutes followed by rinsing 5 times each with SSC/HEPES/SDS (60 ul) and HEPES/NaCl (60 ul). The scavenger is applied again in the manner described above, and the slide is again imaged to determine the effectiveness of the cleave/cap steps and to identify non-incorporated fluorescent objects.

Where the nucleotide analog includes an optional phosphate group in place of the OH group in the 3′ position of the sugar, the phosphate group is removed using alkaline phosphatase. The alkaline phosphatase is then either washed away as described above or is heat inactivated by heating the flow cell to a suitable temperature for a suitable period of time. The optional phosphate group can be removed prior to detection, after detection, after removal of the fluorescent label or after taking the final image to determine the effectiveness of the cleave/cap steps.

The procedure described above is then conducted 100 nM Cy5dATP analog, followed by 100 nM Cy5dGTP analog, and finally 500 nM Cy5dUTP, each as described above. The procedure (expose to nucleotide, polymerase, rinse, scavenger, image, rinse, cleave, rinse, cap, rinse, scavenger, final image, removal of optional phosphate group) is repeated exactly as described for ATP, GTP, and UTP except that Cy5dUTP is incubated for 5 minutes instead of 2 minutes. Uridine is used instead of thymidine due to the fact that the Cy5 label is incorporated at the position normally occupied by the methyl group in thymidine triphosphate, thus turning the dTTP into dUTP. In all 64 cycles (C, A, G, U) are conducted as described in this and the preceding paragraph.

Once 64 cycles are completed, the image stack data (i.e., the single molecule sequences obtained from the various surface-bound duplex) is aligned to the M13 reference sequence. The image data obtained is compressed to collapse homopolymeric regions. Thus, the sequence “TCAAAGC” would be represented as “TCAGC” in the data tags used for alignment. Similarly, homopolymeric regions in the reference sequence are collapsed for alignment.

The alignment algorithm matches sequences obtained as described above with the actual M13 linear sequence. Placement of obtained sequence on M13 is based upon the best match between the obtained sequence and a portion of M13 of the same length, taking into consideration 0, 1, or 2 possible errors. All obtained 9-mers with 0 errors (meaning that they exactly matched a 9-mer in the M13 reference sequence) are first aligned with M13. Then 10-, 11-, and 12-mers with 0 or 1 error are aligned. Finally, all 13-mers or greater with 0, 1, or 2 errors are aligned.

All publications, patents, and patent applications cited herein are hereby expressly incorporated by reference in their entirety and for all purposes to the same extent as if each was so individually denoted. The patent application entitled “Nucleotide Analogs” filed on even date herewith (Attorney Docket Number: HEL-033) is expressly incorporated by reference.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

We claim:

1. A nucleotide analog having the structure: 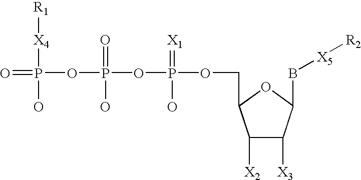
wherein X₁ is O or S;
X₂ is OH or PO₄;
X₃ is H or OH;
R₁ is D or Q, wherein
D is a detectable moiety, and
Q is a quenching moiety capable of modulating signal produced by a detectable moiety;
where R₁ is D, R₂ is Q capable of modulating signal produced by D,
where R₁ comprises Q, R₂ is D;
X₄ is selected from the group consisting of O, N, and S;
B is selected from the group consisting of a purine, a pyrimidine and derivatives thereof, and
R₂ is linked to B by a cleavable linkage X₅.

2. The nucleotide analog of claim 1, wherein B is selected from the group consisting of cytosine, uracil, thymine, adenine, guanine, and analogs thereof.

3. The nucleotide analog of claim 1, wherein the detectable moiety D is selected from the group consisting of fluorescein, BODIPY, EDANS, rhodamine, Cy3, Cy5, and derivatives thereof.

4. The nucleotide analog of claim 1, wherein the quenching moiety Q is selected from the group consisting of fluorescein, BODIPY, EDANS, rhodamine, Cy3, Cy5, DABCYL, and derivatives thereof.

5. The nucleotide analog of claim 1, wherein the cleavable linkage X₅ is a chemically cleavable linkage.

6. The nucleotide analog of claim 1, wherein the chemically cleavable linkage is a disulfide bond.

7. The nucleotide analog of claim 1, wherein the cleavable linkage X₅ is a photochemically cleavable linkage.

8. The nucleotide analog of claim 7, wherein the photochemically cleavable linkage is selected from the group consisting of o-nitrobenzyl and derivatives thereof.

9. The nucleotide analog of claim 1, wherein the cleavable linkage is selected from the group consisting of 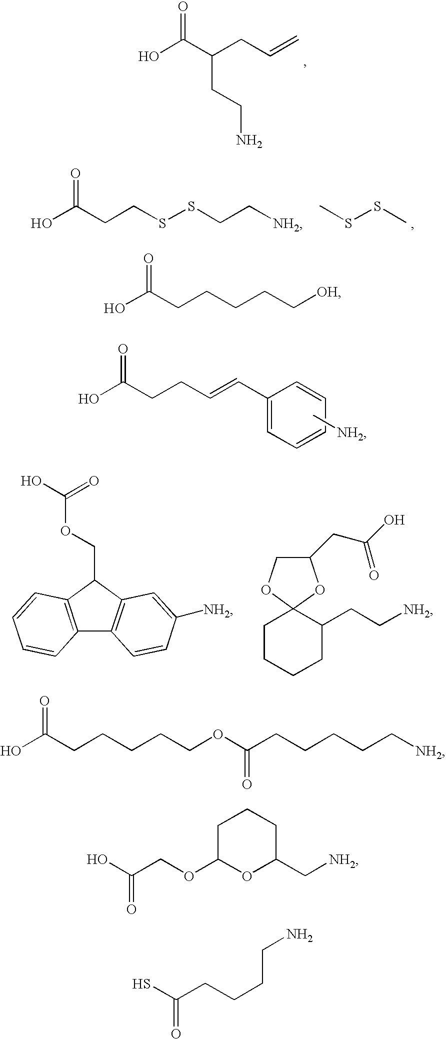
and derivatives thereof.

10. The nucleotide analog of claim 1, wherein X₁ is S.

11. The nucleotide analog of claim 1, wherein X₂ is PO₄.

12. The nucleotide analog of claim 10, wherein X₂ is PO₄.

13. A method for nucleic acid sequence determination, comprising the steps of:
a) exposing a target nucleic acid to a primer that is complementary to at least a portion of the target nucleic acid under conditions suitable for hybridizing the primer to the target nucleic acid, a nucleotide analog of claim 1, and a polymerizing agent, under conditions suitable for extending the primer in a template-dependent manner;
b) detecting incorporation of a nucleotide in each extended primer;
c) treating each hybridized extended primer of b) such that R₂ is removed; and
d) repeating steps a), b) and c), thereby determining the sequence of the target nucleic acid.

14. The method of claim 13, R₂ being removed photochemically.

15. The method of claim 13, R₂ being removed by treating the hybridized extended primer with a reducing agent.

16. The method of claim 15, the reducing agent being selected from the group consisting of dithiothreitol and tris(2-carboxyethyl) phosphine hydrochloride.

17. The method of claim 15, further comprising treating the hybridized extended primer with a capping agent.

18. The method of claim 17, the capping agent being iodoacetamide.

19. The method of claim 13, wherein X₂ is PO₄, the method further comprising treating the hybridized extended primer of b) with an enzyme to remove said PO₄, such that the extended primer can be further extended in subsequent steps.

20. The method of claim 13, wherein said target nucleic acid is attached to a substrate.

21. The method of claim 13, wherein said target nucleic acid is individually optically resolvable.
